(19) United States
(12) Patent Application Publication
(10) Pub. No.: US 2025/0179427 A1
(43) Pub. Date: Jun. 5, 2025

(54) METHOD AND CULTURE MEDIUM FORMULATION WHICH CAN BE USED TO OBTAIN MOTOR NEURONS FROM PLURIPOTENT STEM CELL-DERIVED NEUROMESODERMAL STEM CELLS

(71) Applicant: YEDITEPE UNIVERSITESI, Istanbul (TR)

(72) Inventors: Fikrettin SAHIN, Istanbul (TR); Aysegul DOGAN, Istanbul (TR); Selinay SENKAL, Istanbul (TR)

(73) Assignee: YEDITEPE UNIVERSITESI, Istanbul (TR)

(21) Appl. No.: 18/843,330

(22) PCT Filed: Mar. 2, 2023
(86) PCT No.: PCT/TR2023/050203

§ 371 Date: Sep. 3, 2024

(30) Foreign Application Priority Data
Mar. 3, 2022 (TR) 2022/003171

(51) Int. Cl. C12N 5/0793; C12N 5/00

(52) U.S. Cl. CPC C12N 5/0619; C12N 5/0037; C12N 2501/11; C12N 2501/115; C12N 2501/385; C12N 2501/41; C12N 2501/48; C12N 2501/727; C12N 2506/45; C12N 2533/54; C12N 2533/90

(57) ABSTRACT

A culture medium and a method using the said medium for obtaining motor neuron cells from human pluripotent stem cell-derived neuromesodermal stem cells (NMSCs) are provided. Furthermore, the present invention relates to obtaining motor neuron cells which can be used in research and clinical applications thanks to the said culture medium and method.

### CROSS REFERENCE TO THE RELATED APPLICATIONS

This application is the national phase entry of International Application No. PCT/TR2023/050203, filed on Mar. 2, 2023, which is based upon and claims priority to Turkish Patent Application No. 2022/003171, filed on Mar. 3, 2022, the entire contents of which are incorporated herein by reference.

### TECHNICAL FIELD

The present invention relates to a culture medium and a method using the said medium for obtaining motor neuron cells from human pluripotent stem cell-derived neuromesodermal stem cells (NMSCs). Furthermore, the present invention relates to obtaining motor neuron cells which can be used in research and clinical applications thanks to the said culture medium and method.

### BACKGROUND

The spinal nerves in the spine are part of the central nervous system. They enable nerve conduction between the brain and the body, allowing autonomic functions such as muscle movement, sensory conduction and digestion to take place. Spinal cord injury occurs as a result of mechanical damage. Inflammation and cell death at the injured site cause severe neuronal injury [1]. Neuronal injury to the spinal cord results in irreversible dysfunction. In spinal cord injury cases, functional recovery can be observed at a very low rate as 2.1% [2].

Spinal cord neurons are part of the central nervous system and have poor intrinsic regenerative capacity. This limits the treatment options. Although the contribution of the axonal elongation of neuron cells at the injured site to the tissue regeneration allows limited regeneration, functional tissue regeneration is still low [3]. In this context, transplanting new neurons or cells which can differentiate into neuron cells into the injury site to ensure nerve conduction in the site is a cellular therapy method. In previous studies reported in the literature, although a small portion of transplanted cells could differentiate into nerve cells, it is not sufficient and is not considered functional [4]. For this reason, it is very important to obtain functional neurons, preserve them in culture and enable nerve conduction. Obtaining the source from which cells will be derived and the functional cells in the culture is very important in this context.

Pluripotent stem cells are classified as embryonic stem cells and induced pluripotent stem cells and they can differentiate into many cell types thanks to their pluripotent properties. Several attempts have been made to use pluripotent human stem cells which can differentiate into all cells, as a stem cell therapy product. In the literature, the following patent documents are provided with regard to obtaining spinal cord neurons from pluripotent stem cells and the use of neuromesodermal stem cells as an intermediate step. We would like to state that, considering the information in the prior art, there is no culture medium and no protocol similar to the one described herein in the present invention. The patent documents mentioned below are different inventions in the field of obtaining functional neurons.

The patent documents known in the state of the art are as follows: U.S. Pat. No. 7,531,354B2 WO2009122413A1, WO2014/148646, US20170283774A1, US20160068806A1.

### SUMMARY

The present invention relates to a culture medium and a method using the said medium for obtaining functional neuron from pluripotent stem cells by using the neuromesodermal stem cell (NMSC) as an intermediate step.

The present invention relates to a culture medium and a method using the said medium for obtaining motor neuron cells from human pluripotent stem cell-derived neuromesodermal stem cells.

The present invention relates to a culture medium and a method using the said medium, which overcomes all of the above-mentioned problems and provides additional advantages to the relevant technical field and allows for the unlimited acquisition of motor neurons, which cannot be obtained from adult individuals under normal conditions, from pluripotent stem cells.

The said method disclosed in the invention allows motor neurons to be obtained functionally from neuromesodermal stem cells, which is a developmental biology step of motor neurons in culture. It also allows for the repeated derivation of motor neurons from pluripotent stem cells.

The present invention relates to a culture medium in which motor neurons can be passaged in culture for a long period of time, and to a method using the said medium.

The developed method and the content of the culture medium formulation can be implemented for motor neuron differentiation of embryonic stem cells obtained for research purposes or as a result of therapeutic cloning, and of induced pluripotent stem cells obtained by reprogramming, through neuromesodermal stem cell as intermediate step.

The developed method and the content of the culture medium formulation can be used for reprogramming and culturing of cells obtained from adult individuals, and for the differentiation of neuromesodermal stem cells and motor neurons.

The developed method and the content of the culture medium formulation are designed to be applied in a time-dependent manner by being made in the form of a kit for triggering the differentiation. The present invention can be applied as a kit, formulation, protocol or cell source.

The developed method and the content of the culture medium formulation can be used in a personalized manner or by being adapted to GMP conditions for cellular therapy.

The developed method and the content of the culture medium formulation can be used in motor neuron therapy, nerve injury disease treatments, neurodegenerative disease treatments and nerve injury treatments in general.

The invention relates to the use of a culture medium at specific time intervals in method steps.

The said method disclosed in the present invention ensures that motor neuron cells are obtained by means of a culture medium at certain time intervals and their proliferation is provided while preserving their characteristics.

The present invention also relates to the formulation of a culture medium which can be used for differentiating motor neuron cells from human pluripotent stem cell-derived neuromesodermal stem cells.

The said invention can be used in all centers that carry out research-related (R&D, P&D) processes (universities, research institutes, private sector and company-based organizations that conduct research), companies that prepare and commercially market products for cellular therapy for research and application, centers that carry out processes related to Clinical Practices (GMP) (university hospitals, universities, GMP centers), all centers that carry out processes related to health applications, hospitals, as well as in clinics and hospitals that can apply cellular therapy, indirectly in cooperation with the above-mentioned centers in case they use stem cell-derived material and providing services for the treatment and rehabilitation of nervous system diseases such as spinal cord injury, neuron injury.

### BRIEF DESCRIPTION OF THE DRAWINGS

“A Method and Culture Medium Formulation Which Can Be Used to Obtain Motor Neurons From Pluripotent Stem Cell-Derived Neuromesodermal Stem Cells”, which was performed for achieving the objective of the present invention, is illustrated in the accompanying Figures, in which:

FIG. 1 Derivation of NMSCs from induced pluripotent stem cells and their characterization. Derivation of NMS cells is performed in 3-day suspended culture medium by using bFGF and CHIR. Cells obtained at the end of three days show positive staining for T/Bra and Sox-2 proteins, which are NMSC markers. The resulting cells are NMS cells. Notes: bFGF (basic fibroblast growth factor), CHIR (CHIR99021-Wnt pathway activator), T/Bra (Brachyury), Sox-2 (SRY (sex determining region Y)-box 2).

FIGS. 2A-2B Protocol steps of obtaining spinal cord neurons from induced pluripotent stem cells and the obtained neurons. The said protocol was carried out in N2B27 medium using specific factors as indicated in FIG. 2A. After the fifteenth day, active neurons were obtained and showed activity. In FIG. 2B, the formed neurons extending out of the embryoid bodies are observed in a time-dependent manner. As a result, the implemented protocol functioned actively, and neurons were obtained. Notes: bFGF (basic fibroblast growth factor), CHIR (CHIR99021-Wnt pathway activator), RA (Retinoic acid), SAG (SAG dihydrochloride—Smoothened Agonist), Shh (Sonic Hedgehog), D: days

FIG. 3 Immunostaining and intensity measurements of neuron markers in the obtained neurons. The neurons obtained were active at the end of fifteen days and their active neuron markers were shown by staining at the protein level. The results of the staining showed presence of B-Tubulin, Isl-1, Isl-2, MNX-1, Neun, NF, Map2 proteins, which were observed in neurons. The resulting neurons have motor neuron characteristics.

FIG. 4 Neurons rapidly attaching to the surface after freeze-thawing. A freeze-thaw study was performed to show that the obtained neurons in culture can be cryopreserved. The neurons quickly attached to the surface after thawing and there was no morphological change. As a result, the obtained motor neurons can be cryopreserved in culture.

FIGS. 5A-5C Axonal elongation of the obtained spinal nerves. When the neurons obtained as a result of the protocol application are monitored from the same region for a period of 3 days, axonal elongation and the dynamic structure of the neurons can be seen. Neurons elongate and form connections. As a result, neurons were observed to perform axonal elongation and form synapses (connections between neurons). Notes: D (days)

FIGS. 6A-6B Synaptic vesicles and neuron transmission. It was observed that the obtained neurons were connected to each other in the form of a network and synaptic vesicles were formed in these connections. This is an indication of active neuron formation and nerve conduction. Arrows indicate the synaptic vesicles.

### DETAILED DESCRIPTION OF THE EMBODIMENTS

The present invention relates to a culture medium and a method using the said medium for obtaining motor neuron cells from human pluripotent stem cell-derived neuromesodermal stem cells.

The present invention comprises motor neuron cells produced by this method, cellular therapy products which can be used in spinal cord injury or neuronal injury applications, and a culturing method and a culture medium, which are standardized, optimized, and can be used in clinical applications, for producing motor neuron cells from human pluripotent stem cells.

As used herein, the term “stem cells” refers to master cells which can be renewed unlimitedly to form the cells of tissues and organs. Stem cells are totipotent, multipotent, pluripotent, oligopotent or unipotent cells.

As used herein, the term “pluripotent stem cells” refers to pluripotent stem cells which can differentiate into all three germ layers that constitute a living body, and examples of these include embryonic stem cells and induced pluripotent stem (iPS) cells. The said method can be adapted to and used with all pluripotent stem cells, including induced pluripotent stem cells and embryonic stem cells. In the preferred embodiment of the invention, human induced pluripotent stem cells are used for obtaining motor neurons through neuromesodermal stem cells.

As used herein, the term “differentiation” refers to a process by which cells become specialized in terms of structure or function during cell division, proliferation and growth, whether in embryonic development or in the adult individual.

As used herein, the term “embryoid body” refers to an aggregate formed by inducing the differentiation of pluripotent stem cells. They are 3-dimensional structures.

In order to treat spinal cord injuries on a cellular basis, neuromesodermal stem cells can differentiate into neuron (nerve) cells in the spinal cord.

The present invention comprises a culture medium formulation and the use of this culture medium in the said method, for the differentiation of motor neuron cells from human pluripotent stem cell-derived neuromesodermal stem cells (NMSCs).

The culture medium formulation of the present invention contains DMEMF12/neurobasal medium (1:1), Bovine Serum Albumin (BSA), N2 supplement, B27 supplement, penicillin (10.000 U/ml), streptomycin (10.000 g/ml) and L-glutamine in N2B27 serum-free medium, and it additionally contains bFGF (basic fibroblast growth factor), and CHIR-99021 (Wnt activator).

In the preferred embodiment of the invention, the concentration of bFGF in the culture medium is 12 ng/ml and the concentration of CHIR-99021 is 3 μM. The specified doses are fixed and should be used accordingly.

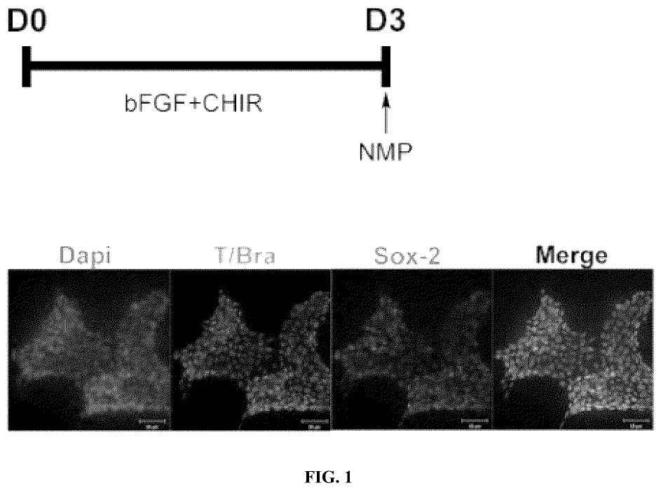

In the serum-free culture medium of the present invention, induced pluripotent stem cells were treated with 12 ng/ml bFGF and 3 μM CHIR-99021 for three days and differentiated into neuromesodermal stem cells (NMSCs) (FIG. 1). This differentiation was performed in suspended culture medium and embryoid bodies were obtained.

The NMSC phenotype of differentiated cells was demonstrated and proven by T/Bra and Sox-2 staining (FIG. 1).

In the preferred embodiment of the invention, the culture medium formulation contains DMEMF12:neurobasal medium (1:1), 0.5% Bovine Serum Albumin (BSA), 0.5% N2 supplement, 1% B27 supplement, 1% penicillin (10.000 U/ml)-streptomycin (10.000 g/ml) and 1% L-glutamine in N2B27 serum-free medium.

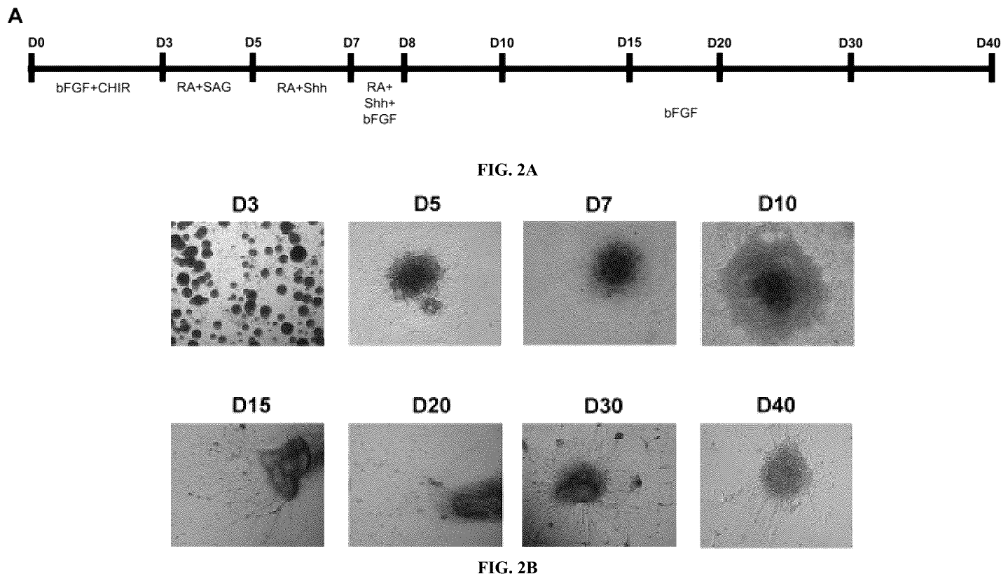

Neuromesodermal stem cells were differentiated into spinal cord neurons in culture medium containing N2B27 (serum-free medium) as shown in FIG. 2A. The time-dependent steps used in the method and the medium content applied therein are shown in Table 1.

TABLE 1
Method steps and content

| Time | Differentiation method |
|---|---|
| Day 0-Day 3 | 12 ng/ml bFGF + 3 uM CHIR |
| Day 3-Day 5 | 0.1 uM RA + 0.5 uM SAG |
| Day 5-Day 7 | 0.1 uM RA + 50 ng/ml Shh |
| Day 7-Day 8 | 0.1 uM RA + 50 ng/ml Shh + 10 ng/ml bFGF |
| Day 8 and onwards | 10 ng/ml bFGF |

The obtained neurons had an active neuron phenotype by day 15 (FIG. 2B). These neurons could be preserved in culture for up to 40 days by passaging with the scraping method. This makes it possible to preserve neurons in culture. The cells forming the embryoid bodies in suspended culture until the third day were transferred to the surface coated with matrigel-gelatin mixture on the third day, thereby attaching thereto, and the culturing was then carried out in two-dimensional culture.

As used herein, the expression “culture medium” includes culture media free of bFGF, CHIR, RA, SAG, and Shh.

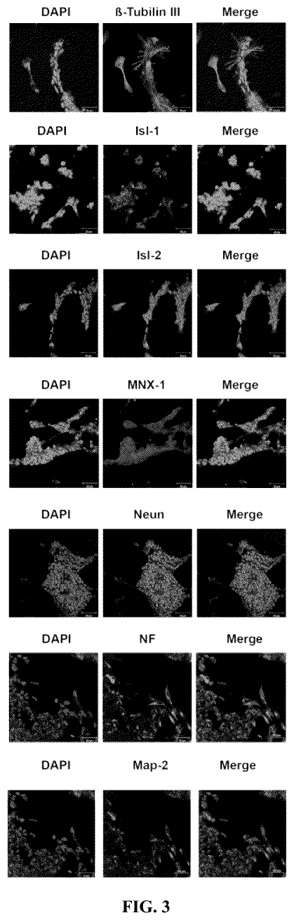

The obtained neurons were immunostained with P-Tubulin, Isl-1, Isl-2, Minx1, NeunN, NF, Map-2 markers and characterized by their neuronal properties. FIG. 3 shows the immunostaining with neuron markers.

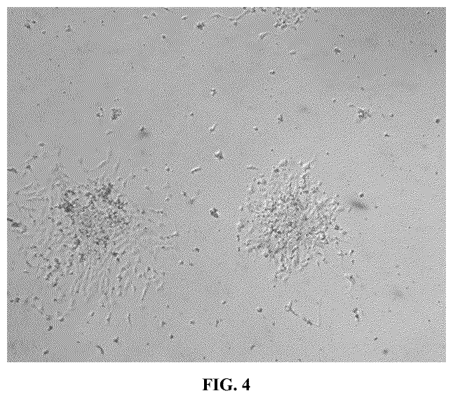

The obtained neurons were freeze-thawed with a solution obtained with the addition of 10% DMSO to N2B27 medium which contains bFGF and is also a culture medium, and they were able to maintain their viability (FIG. 4). Freezing was performed by scraping and precipitating the cells and suspending them in freezing solution. The prepared vials were kept in a tank with isopropanol at −80 degrees Celsius overnight and then stored in a nitrogen tank at −196 degrees Celsius.

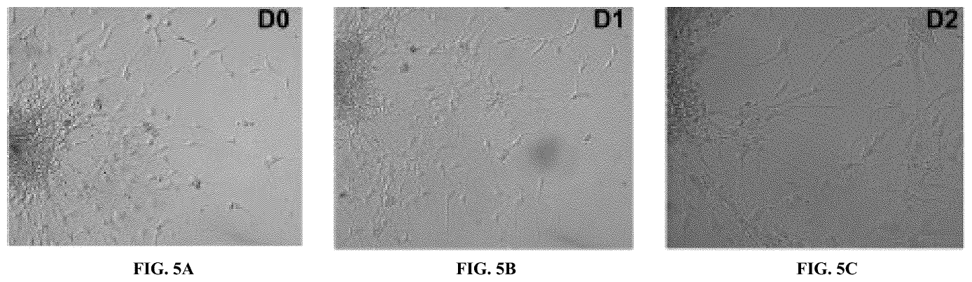

The obtained neurons have a dynamic structure in culture and axonal elongation occurs. This is the first indication that neurons show functional activity (FIGS. 5A-5C).

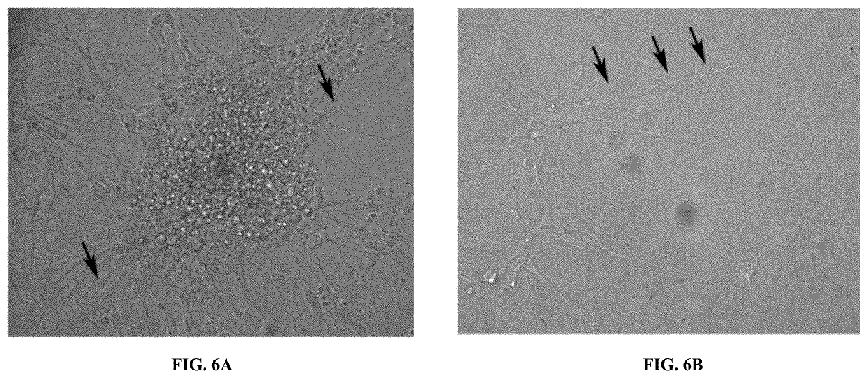

In the obtained neurons, synaptic vesicles indicating nerve conduction between axons and conduction in axons were observed. This is an indication of functional neuron activity (FIGS. 6A-6B).

The present invention relates to a method for culturing the stem cells by using a culture medium to differentiate human pluripotent stem cells into motor neurons through neuromesodermal stem cells (NMSCs) and comprises the following steps:
a) differentiating the induced human pluripotent stem cells into neuromesodermal stem cells (NMSCs) by treatment with bFGF and CHIR-99021 in a suspended serum-free N2B27 culture medium;
b) attaching the formed neuromesodermal stem cells to a surface in the culture medium;
c) adding, respectively, RA and SAG; RA, Shh and bEGF; bEGF in serum-free N2B27 culture medium in order to ensure that the attached neuromesodermal stem cells differentiate into functional motor neuron cells.

In step (a) of the method of the present invention, the induced human pluripotent stem cells are cultured in serum-free N2B27 culture medium, which is prepared with the addition of 12 ng/ml bFGF and 3 μM CHIR-99021, and in suspended culture in an incubator with a temperature of 37° C. and %5 CO2 atmosphere for 3 days. (FIG. 1)

In step (b) of the method of the present invention, the neuromesodermal stem cells formed in step (a) are attached to a surface. This step allows the transfer of neuromesodermal stem cells from 3-dimensional culture to 2-dimensional culture. The cells, which are obtained in suspended culture until the third day were transferred to the surface coated with matrigel-gelatin mixture on the third day, thereby attaching thereto, and the culturing was then carried out in two-dimensional culture. Thereby, the cells adhere to the surface after 3-day suspended culture and perform differentiation (FIG. 1).

After the attaching process in step (b), in step (c) of the method of the present invention, the culturing process is continued by adding 0.1 uM RA+0.5 uM SAG between days 3 to 5, 0.1 uM RA+50 ng/ml Shh between days 5 to 7 and 0.1 uM RA+50 ng/ml Shh+10 ng/ml bFGF between days 7 to 8 into N2B27 culture medium. After incubation until day 8, culture medium with addition of 10 ng/ml bFGF is introduced.

In this step (c), cell differentiation continued until day 15 and the obtained neurons had an active neuron phenotype by day 15. In this way, transition of the cells transit to a functional motor neuron cell phenotype was aimed. The protocol steps have such transitions. These neurons can be passaged by scraping method and preserved in culture from day 15 to day 40, and the method is a protocol in which active neurons are obtained on day 15 and can be preserved up to 40 days in total (FIG. 1).

Motor neuron cells produced by the method of the present invention have characteristics which can be used in cell therapy for spinal cord injuries or neuronal injuries.

The following examples are to better illustrate the subject of the invention and the subject of the invention is not limited to these examples.

### EXAMPLES

### Example 1

Technique for the derivation of neuromesodermal cells: Within the scope of this invention, neuromesodermal stem cells were used as a preliminary step to obtain neurons. Technically, these cells were obtained by keeping the pluripotent stem cells in suspended culture for three days in medium containing bFGF and CHIR without attachment (FIG. 1). NMS cells were thus obtained as embryoid bodies (FIGS. 2A-2B). They, like their in vivo counterparts, NMS cells, express T/Bra and Sox2 factors simultaneously (FIG. 1).

Obtaining neurons from NMS cells: Although NMS cells were obtained in suspended medium, neuronal differentiation of these cells was performed in monolayer cell culture medium. Embryoid bodies, which were transferred to surfaces coated with matrigel-gelatin mixture, adhered to the surfaces, wherein they were subjected to the differentiation protocol in FIGS. 2A-2B and neurons were formed.

The formed neurons elongate their axons throughout the differentiation program, showing active neuron morphology (FIGS. 2A-2B and FIGS. 5A-5C) and perform neuron transmission through lipid vesicle movements (FIGS. 6A-6B).

### REFERENCES

[1]. Zhang, N., Yin, Y., Xu, S. J., Wu, Y. P., Chen, W. S. 2012. “Inflammation & apoptosis in spinal cord injury”, The Indian Journal of Medical Research, 135(3), 287-296.
[2]. Shroff, G. 2016. “Human embryonic stem cell therapy in chronic spinal cord injury: a retrospective study”, Clinical and Translational Science, 9(3), 168-175.
[3]. Dell'Anno, M. T., Strittmatter, S. M. 2016. “Rewiring the spinal cord: direct and indirect strategies”, Neuroscience Letters, 652, 25-34.
[4]. Abematsu, M., Smith, I., Nakashima, K. 2006. “Mechanisms of neural stem cell fate determination: extracellular cues and intracellular programs”, Current Stem Cell Research & Therapy, 1, 267-277.

What is claimed is:

1. A method for culturing stem cells by using a culture medium to differentiate human pluripotent stem cells into motor neurons through neuromesodermal stem cells (NMSCs), comprising the following steps:
a) differentiating induced human pluripotent stem cells into the NMSCs by a treatment with bFGF and CHIR-99021 in a suspended serum-free N2B27 culture medium;
b) attaching the NMSCs formed to a surface in the suspended serum-free N2B27 culture medium;
c) adding, respectively, RA and SAG; RA, Shh, and bEGF to the suspended serum-free N2B27 culture medium; wherein the bEGF in suspended serum-free N2B27 culture medium ensures attached NMSCs differentiate into functional motor neuron cells.

2. The method according to claim 1, wherein the step a) continues until day 3, the step b) continues on day 3, and the step c) continues from day 3 until days 8 to 40.

3. The method according to claim 2, wherein the method lasts 15 to 40 days in total.

4. The method according to claim 3, wherein in the step a), a concentration of the bFGF is 12 ng/ml and a concentration of CHIR-99021 is 3 μM.

5. The method according to claim 4, wherein the suspended serum-free N2B27 culture medium in the step a) is a N2B27 serum-free medium comprising DMEMF12:neurobasal medium (1:1), 0.5% Bovine Serum Albumin (BSA), 0.5% N2 supplement, 1% B27 supplement, 1% penicillin (10.000 U/ml)-streptomycin (10.000 g/ml), and 1% L-glutamine.

6. The method according to claim 5, wherein in the step c),supplements are added in the suspended serum-free N2B27 culture medium as follows:
adding 0.1 uM RA and 0.5 uM SAG between days 3 to 5;
adding 0.1 uM RA and 50 ng/ml Shh between days 5 to 7;
adding 0.1 uM RA, 50 ng/ml Shh, and 10 ng/ml bFGF between days 7 to 8; and
adding 10 ng/ml bFGF between days 8 to 40.
